(19) United States
(12) Patent Application Publication
(10) Pub. No.: US 2023/0139105 A1
(43) Pub. Date: May 4, 2023

(54) CIRCUMAURAL EAR CUSHION/SEAL

(71) Applicant: LIGHTSPEED AVIATION, INC., Lake Oswego, OR (US)

(72) Inventors: Allan SCHRADER, Lake Oswego, OR (US); Brian David FROST, Lake Oswego, OR (US)

(73) Assignee: LIGHTSPEED AVIATION, INC., Lake Oswego, OR (US)

(21) Appl. No.: 17/979,409

(22) Filed: Nov. 2, 2022

Related U.S. Application Data
(60) Provisional application No. 63/274,935, filed on Nov. 2, 2021.

(51) Int. Cl. A42B 3/16; A42B 3/06; H04R 1/10

(52) U.S. Cl. CPC A42B 3/163; A42B 3/06; H04R 1/1083; H04R 1/1008

(57) ABSTRACT

A helmet includes a shell configured to extend over ears of a user with integrated acoustic ear cups having a polygonal cushion/seal formed by an upper hexagonal portion connected to a lower inverted triangular portion with a wedge-shaped profile/thickness thinner in the upper portion than at least some of the lower portion to provide non-uniform side loading that improves wearer comfort and reduces acoustic leak paths for passive or active noise reduction (ANR). A headband, headset, or earmuffs for hearing protection include a polygonal cushion/seal with a wedge-shaped profile/thickness to facilitate acoustic sealing and user comfort.

### CROSS-REFERENCE TO RELATED APPLICATION

This application claims the benefit under 35 U.S.C. § 119(e) to U.S. Provisional Application Ser. No. 63/274,935 filed Nov. 2, 2021, the disclosure of which is hereby incorporated by reference in its entirety.

### TECHNICAL FIELD

This application relates to a circumaural ear cushion/seal for audio communications, active noise reduction, hearing protection, or similar applications that may use a helmet, headset, headband or similar device to position an ear cup over the ear of a user.

### BACKGROUND

Helmets are worn to protect the head of the user from injuries that may occur in a wide variety of recreational, occupational, transportation, and military applications. The helmet design may vary depending on the type and frequency of expected impacts. Similarly, use patterns may vary from repeated removal of the helmet between short duration uses, such as those that may occur in football or hockey, to extended periods of use, such as those that may occur in occupational, transportation, or military applications, for example. In many applications, the helmet may extend partially or completely over the ears of the user. The helmet may be designed to reduce transmission of external sound, or to minimally impact transmission of external sound to the wearer. Active audio devices such as speakers and microphones and/or passive devices such as acoustic sound absorbing material may be used alone or in combination to provide the desired helmet acoustics.

In passive and active noise reduction helmet applications, performance and wearability may be improved by providing a complete seal around the ears without compromising comfort over long use durations. Current helmet designs appear to lack the ability to achieve these seal and comfort goals without significant tradeoffs between them.

A variety of non-military helmets and others that do not include fully customized shells provide adjustable ear cups. However, the ear cup assembly is mounted and positioned within the helmet using a repositionable or removable fastener, such as a hook and loop closure. The external shell is a hard molded shape with the ear cup assembly moveable within the inner lining of the shell. Foam pads and strips of hook and loop closure material is used to provide a customized fit for each user. These types of positioning systems require that the helmet be removed to position the ear cup assembly and comfortable positioning often requires several trial-and-error attempts by the user. These systems are also generally fixed or static once positioned within the helmet, although repeatedly wearing and removing the helmet may disturb the positioning of the ear cups. In addition, side pressure is established by the external shell dimension and the selection or combination of padding positioned through this iterative process.

Helmets having active noise reduction (ANR) technology to cancel at least some of the unwanted external noise rely on a good seal around each ear to achieve best results. A good seal is particularly difficult to achieve inside a helmet for at least two reasons: helmets generally fit fairly tightly to provide their protective function, and the ear pinna protrudes from the surrounding surfaces of the head and varies in shapes and sizes among users. As such, donning the helmet and proper positioning of previously placed ear cups or earphones can be very challenging. After the helmet is placed on the head, the seal around the ear may not be ideal based on ear position (within the ear cup) or the ear cup position relative to the skull. To achieve desired acoustic performance, users may over-compensate for acoustic leak paths by increasing the side pressure, which may result in reduced comfort, particularly over long periods of time.

For best performance of an ANR system, the positioning of the ear canal and pinna relative to the driver/speaker and ANR feedback microphone within the ear cup or earphones should be understood and repeatable. Current solutions generally fail to deliver consistent performance and comfort with either the acoustics or the cushion/seal system. Largely due to positioning and comfort constraints previously described, existing helmets use a full round/oval seal. Although slot seals may reduce circumference/perimeter distance and result in greater comfort, slot seals may be unsuitable for many helmet applications due to the difficulty in positioning the pinna into the ear slot seal when donning the helmet due to the fixed ear cup within the helmet. Existing ear cushion/seals may apply a uniform pressure distribution that may contribute to discomfort while also creating acoustic leak paths. Furthermore, helmets without independent suspension systems do not improve sealing performance in response to downward pressure from tightening a chin strap, for example.

While helmet applications may present unique challenges for an ear cushion/seal to provide desired ANR and/or hearing protection performance, similar challenges often exist in other form factors, such as headbands and headsets used in communication and hearing protection applications for aviation and industrial applications, for example.

### SUMMARY

In one or more embodiments, a helmet includes a shell configured to extend over ears of a user, an ear cup disposed within the shell, and an ear cushion/seal connected to the ear cup, the ear cushion/seal having a non-uniform shape to reduce circumferential distance below the ear to reduce side loading pressure while allowing positioning of the ear pinna of a user behind the cushion/seal and within the ear cup. The non-uniform shape comprises an annular elongated inverted pentagon or alternatively an annular polygon formed by the upper half of a hexagon connected to an inverted triangle with both the hexagon and triangle having rounded vertices. The polygonal shape includes a wedge-shaped profile or thickness being thinner around the hexagonal portion with increasing thickness extending to the apex of the inverted triangular portion. The non-uniform shape reduces circumferential distance particularly below the ear of a user in the range of about 105° to about 210° (3:30-7:00) clockwise circumference relative to the top vertical position of the ear of a user. The non-uniform shape reduces side loading pressure while allowing positioning of the pinna within an ear cup relative to more conventional oval or round cushions/seals.

In one embodiment, a helmet having a shell configured to receive and substantially surround integrated circumaural ear cups includes a polygonal ear cushion secured to each ear cup, each ear cushion defining a hexagonal upper portion connected to an inverted triangular lower portion with both upper and lower portions having radiused or rounded vertices, the ear cushion having an increased thickness around at least some of the lower portion relative to the upper portion.

Various embodiments may include a helmet having integrated ear cups with an ear cushion/seal as previously described with one or more of the ear cups including active noise reduction (ANR) components, which may include one or more microphones, speakers/drivers, programmed microprocessors, memory and/or electronics to provide active noise reduction.

Embodiments according to the present disclosure may have one or more associated advantages. For example, a helmet having integrated ear cups with a polygonal ear cushion/seal according to this disclosure may provide improved ANR performance, passive noise reduction, and comfort relative to conventional oval or round cushions. Reduced circumferential distance particularly below the ear according to one or more embodiments reduces side loading pressure. The polygonal ear cushion also facilitates placement of a biometric sensor over the temporal superficial artery (TSA) forward of the seal, eliminating the need to integrate the sensor into the seal for proper TSA placement.

The above advantages and other advantages and features will be readily apparent from the following detailed description when taken in connection with the accompanying drawings.

### BRIEF DESCRIPTION OF THE DRAWINGS

FIG. 1 is a side view of a helmet having integrated ear cups and a polygonal ear cushion/seal according to various embodiments.

FIG. 2 is a plan view of a representative embodiment of a polygonal ear cushion/seal for various ear cup applications, including ANR, hearing protection, and communications for helmets, headbands, and headsets.

FIG. 3 is a side view of a representative embodiment of a polygonal ear cushion/seal illustrating a wedge-shaped profile/thickness with increased thickness in at least some of the inverted triangular lower portion.

FIG. 4 illustrates a polygonal ear cushion/seal installed on an ear cup having ANR components.

FIG. 5A illustrates a circumference/perimeter distance of a representative prior art slot seal design.

FIG. 5B illustrates a circumference/perimeter distance of a representative prior art oval seal design.

FIG. 5C illustrates a circumference/perimeter distance of a representative embodiment of a seal design according to the present disclosure.

### DETAILED DESCRIPTION

As required, detailed embodiments are disclosed herein; however, it is to be understood that the disclosed embodiments are merely exemplary and may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the teachings and representative embodiments of the disclosure.

For ease of description and illustration, this disclosure may use terms of relative motion that are intended to be interpreted broadly with respect to a helmet as normally worn on the head of a user. Alternatively, movement may be described along x, y, and z axes assigned according to an industry standard, such as SAE J211, for example. As such, terms such as inward and outward refer to movement toward or away from the head of the user, respectively, or along the y-axis. Similarly, inward and outward movements may be interpreted as movement within the helmet away from the helmet shell and toward the helmet shell, respectively. Other directional terms such as forward or rearward, or movement along the x-axis, may be used to describe directions toward the front or rear of the head of a user wearing the helmet. In a similar manner, upward and downward movements refer to movements along the z-axis toward the crown or chin, respectively.

Circumaural ear cups deliver a level of noise attenuation as a primary function. The cup structure (shape, materials, thickness, volume) all contribute to the maximum theoretical or ideal attenuation of the system. The ear cushion/seal includes a flexible membrane attached to the opening of the ear cup, that provides a way to comfortably interface the cup system to a wearer's head. The full 360-degree perimeter or circumference of the cushion/seal must contact the head to seal the space or cavity of the ear cup to the head to provide any meaningful attenuation and noise isolation.

Due to the highly variable shape and contour of a 5%-95% human head, various sizes, thicknesses, and materials are employed to facilitate the desired 360-degree seal. The present inventors have recognized that varying amounts of side pressure is required to ensure the seal will compress and conform to the irregular surface contours of a given head to provide the desired seal. While the relative contours follow a narrower pattern of variability near the attachment of the earform to the headform, the contour variability grows exponentially as the seal surface moves further from the base of the ear. The need for increased side pressure grows as a ‘wider’ and/or longer seal line that is formed by a given ear seal shape. As such, user comfort is directly and significantly correlated with the ear seal circumference/perimeter design.

As also recognized by the present inventors, for a given ear cushion seal design (material, thickness, volume, etc.), smaller circumferential lengths will provide greater comfort for the user. Various seal cover and compression materials, along with the contours of the seal surface, will also affect user comfort. Attention to the design requirements for 5th-95th percentile head and ear form data is also required to maximize comfort. But if all else is equal in comparative comfort, circumferential length will inversely correlate to user comfort, i.e. shorter circumferential length corresponds to increased user comfort.

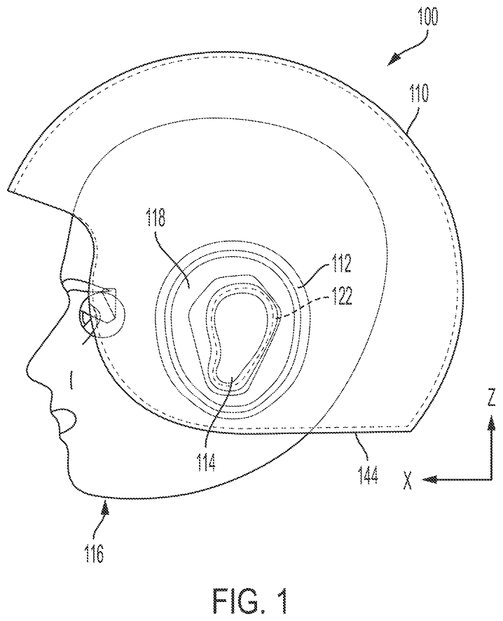

Referring now to FIG. 1, a side view of a helmet having integrated ear cups with polygonal ear cushions/seals according to various embodiments is shown. Helmet 100 includes a shell 110 configured to receive and substantially surround integrated circumaural ear cups 112, only one of which is illustrated in the Figures. Typical applications include an ear cup 112 for each of the ears 114 of a user 116. Each of the ear cups 112 may be substantially identical for left and right ears, or may be customized with different ear cups for the right and left ears 114. Similarly, ear cups 112 may be customized for a particular user with the left and right ear cups for that user being substantially identical. In some embodiments, the left and right ear cups 112 may include different components for passive and/or active noise reduction or audio. For example, in passive noise reduction applications, only one of the ear cups 112 may include a speaker to provide mono audio. Typical active noise reduction (ANR) applications include a driver/speaker and at least one noise-sensing microphone positioned within each of the ear cups 112 to provide active noise reduction and stereo audio for user 116. Helmet 100 may also include an integrated communication microphone (not shown), such as a boom microphone, to capture voice input from user 116.

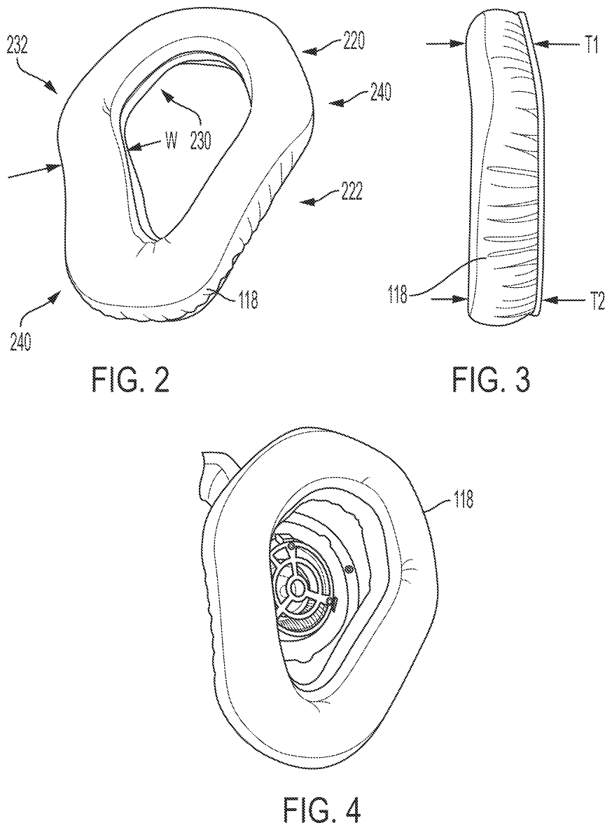

As also shown in FIG. 1, helmet 100 includes a shell 110 configured to cover ears 114 of user 116. In various embodiments, helmet shell 110 substantially or entirely covers ear cups 112 when viewed from the side or along the y-axis. While the front portions of ear cups 112 may be visible from the front and bottom of helmet shell 110, each of the ear cups 112 is contained substantially within shell 110. Ear cups 112 are integrated within shell 110 and each includes a polygonal cushion 118 with a wedge-shaped thickness or profile as described in greater detail with respect to FIGS. 2 and 3. Position of ear cups 112 while helmet 100 is worn by user 116 facilitates positioning of each pinna 122 of an associated ear 114 within the earcup after donning the helmet 100.

FIG. 2 shows a perspective view of a polygonal cushion/seal according to various embodiments. FIG. 3 shows a side or profile view of a polygonal cushion/seal having a wedge-shaped profile according to various embodiments. FIG. 4 shows a perspective view of a polygonal cushion installed on an ANR ear cup according to various embodiments. Referring now to FIGS. 2-4, ear cushion 118 includes a generally hexagonal upper portion 220 connected to an inverted generally triangular lower portion 222. The upper and lower portions may be integrally formed of unitary construction of a closed or open cell foam material to provide cushioning and acoustic sealing around the circumference of an ear of the user. The inner cushion may be surrounded by a generally smooth cover. Various materials may be used to provide the inner cushion and outer cover depending on the particular application and implementation. In the embodiment illustrated, the annular shape of the cushion/seal 118 has a generally uniform width (W) between the inner circumference 230 and the outer circumference 232, and a wedge-shaped profile/thickness (best illustrated in FIG. 3) that is thinner at the upper portion 220 (with thickness T1) and thicker for at least part of the lower portion 222 (with thickness T2). In other embodiments, the width (W) may vary around the circumference. Similarly, the inner circumference may have the polygonal shape illustrated with the outer circumference having a different shape, such as an oval, for example, as generally illustrated in FIG. 1.

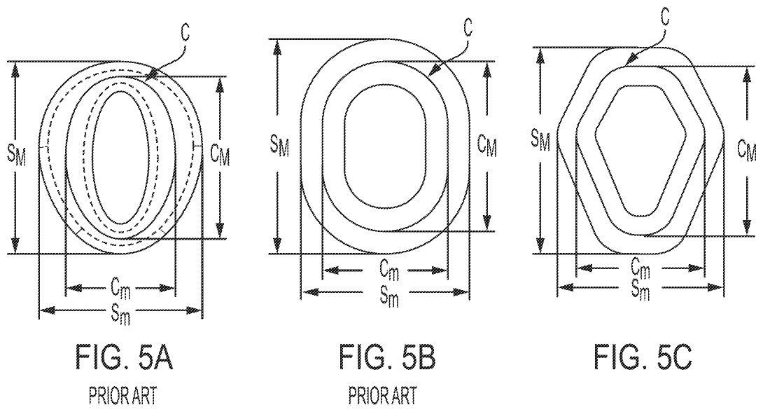

FIG. 5A illustrates a circumference/perimeter distance of a representative prior art slot seal design. FIG. 5B illustrates a circumference/perimeter distance of a representative prior art oval seal design. FIG. 5C illustrates a circumference/perimeter distance of a representative embodiment of a seal design according to the present disclosure. For purposes of comparison, the circumference/perimeter distance referred to herein may be measured along the center or midpoint between the inner and outer circumferences as illustrated in the Figures. As illustrated in the embodiments of FIGS. 1-4, and 5C, the elongated inverted pentagon or alternatively the composite hexagon/triangle shape may have rounded or radiused vertices 240.

The prior art slot-seal design represented in FIG. 5A has a major axis seal dimension S_{M} of 109.55 mm, a minor axis seal dimension Sₘ of 92.91 mm, a circumference/perimeter major axis dimension C_{M} of 92.97 mm, and a circumference/perimeter minor axis Cₘ of 62.45 mm, resulting in a midline circumference/perimeter arc length of 244.6 mm.

The prior art oval design represented in FIG. 5B has a major axis seal dimension S_{M} of 121.00 mm, a minor axis seal dimension Sₘ of 96.00 mm, a circumference/perimeter major axis dimension C_{M} of 95.50 mm, and a circumference/perimeter minor axis Cₘ of 71.00 mm, resulting in a midline circumference/perimeter arc length of 272.0 mm.

The polygonal design with rounded/radiused vertices of one embodiment according to the present disclosure represented in FIG. 5C has a major axis seal dimension S_{M} of 118.10 mm and a minor axis seal dimension Sₘ of 95.10 mm. The resulting midline circumference/perimeter arc length is 264 mm.

The following table provides a relative subjective rating score of design/performance for a seal shape as described and illustrated in the present disclosure as compared to a traditional oval seal design/performance. The rating scale ranges from 1-5 with 1 representing the worst and 5 representing the best.

| | Traditional | Slot | Polygonal- |
|---|---|---|---|
| | Oval Seal | Seal | shaped Seal |
| Side Pressure | 2 | 5 | 4 |
| Required To Seal | | | |
| Ease of Helmet | 5 | 3 | 4 |
| Donning/Doffing | | | |
| TSA Access for | 4 | 2 | 5 |
| Biometric Sensor | | | |

The seal illustrated and described with reference to FIGS. 1-4 and 5C is designed to provide the shortest circumferential length to meet desired performance metrics while providing user comfort and wearability. In various embodiments, the seal is 10-15% shorter than a traditional oval seal, which is designed to fully surround the ear form of a 99th percentile pinna and results in the longest circumferential length of any alternative. While donning of a helmet including a traditional oval seal is very simple, providing a complete seal is often difficult without multiple fitments and increased side pressure. In contrast, the polygonal-type seal according to various embodiments of the disclosure easily accommodates the pinna protrusion of the 95th percentile male. Because the contour variability of the head and particularly jaw line grows significantly as distances from the ear foam base increases, the 10-15% shorter circumferential length provides a 25-30% reduction in needed side pressure for the average wearer.

While a slot-seal design may further reduce the distance around the circumference or perimeter of the seal, the use of a slot-seal design may not be practical for a helmet application. For other applications, the slot-seal design may provide a minimum circumference distance or length obtained from 5%-95% anthropomorphic data that specifies the minimum length, and curvature, to allow such a seal to fit and seal. Due to the variable size/nature of the ear pinna, those data further inform the smallest slot suitable for a comfortable fit of the ear inside the seal. In this case, the ear cup is designed to allow sufficient space for the pinna to fit behind the seal material to insure acceptable wearability. A slot-seal design requires the wearer to insert/position the pinna in and behind the seal material in the space provided. While easier to accommodate on a headphone of a headset, it can be more difficult to properly position when used inside a tight/constrained enclosure of a helmet, which provides primarily a “Y” axis motion for positioning while donning the helmet, whereas the slotted seal requires significant “X” axis mobility to insert the pinna into the slot.

As demonstrated by various embodiments of the present disclosure, a helmet having integrated ear cups with a polygonal seal having a wedge-shaped profile provides non-uniform side loading and reduced circumferential distance to improve acoustic sealing and user comfort while being worn by the user.

While the best mode has been described in detail, those familiar with the art will recognize various alternative designs and embodiments within the scope of the following claims. While various embodiments may have been described as providing advantages or being preferred over other embodiments with respect to one or more desired characteristics, as one skilled in the art is aware, one or more characteristics may be compromised to achieve desired system attributes, which depend on the specific application and implementation. These attributes include, but are not limited to: cost, strength, durability, life cycle cost, marketability, appearance, packaging, size, serviceability, weight, manufacturability, ease of assembly, etc. The embodiments discussed herein that are described as less desirable than other embodiments or prior art implementations with respect to one or more characteristics are not outside the scope of the disclosure and may be desirable for particular applications.

What is claimed is:

1. A helmet, comprising:
a shell configured to extend over ears of a user;
an ear cup disposed within the shell; and
a polygonal cushion having a hexagonal upper portion coupled to an inverted triangular lower portion, the polygonal cushion secured to the ear cup.

2. The helmet of claim 1 wherein the polygonal cushion comprises a wedge-shaped profile being thinner in the hexagonal upper portion than at least some of the inverted triangular lower portion.

3. The helmet of claim 2 further comprising active noise reduction (ANR) circuitry disposed within the ear cup.

4. The helmet of claim 3 wherein the ANR circuitry comprises a speaker/driver and a microphone in communication with a programmed microprocessor, the microprocessor programmed to generate a speaker/driver signal in response to a signal from the microphone.

5. The helmet of claim 2 wherein the polygonal cushion cooperates with the ear cup to form a slot pocket configured to receive a portion of the pinna of an ear of a user donning the helmet.

6. The helmet of claim 1 wherein the polygonal cushion comprises rounded vertices.

7. A wearable device, comprising:
a headband configured to extend around the head of a user;
ear cups connected to each end portion of the headband and configured for positioning over ears of the user; and
a polygonal cushion having a hexagonal upper portion coupled to an inverted triangular lower portion secured to each of the ear cups.

8. The wearable device of claim 7 wherein the polygonal cushion comprises a wedge-shaped profile being thinner in the hexagonal upper portion than at least some of the inverted triangular lower portion.
